(19) United States
(12) Patent Application Publication
(10) Pub. No.: US 2006/0003000 A1
(43) Pub. Date: Jan. 5, 2006

(54) Adhesively bonded dosage form

(76) Inventors: Lawrence Solomon, Boca Raton, FL (US); Allan S. Kaplan, Boca Raton, FL (US)

Correspondence Address: HEDMAN & COSTIGAN P.C., 1185 AVENUE OF THE AMERICAS, NEW YORK, NY, 10036, US

(21) Appl. No.: 11/173,421

(22) Filed: Jul. 1, 2005

Related U.S. Application Data
(60) Provisional application No. 60/584,828, filed on Jul. 1, 2004.

(51) Int. Cl. A61K 9/20

(52) U.S. Cl. 424/464

(57) ABSTRACT

A solid pharmaceutical dosage form having a plurality of adhesively-joined subunits and also having one or more of the following: A. (i) a first inert tablet subunit and (ii) a second active subunit; B. (i) a first tablet subunit with a pharmacologically inactive layer in which said layer has a mass of at least 20 mg and (ii) a second subunit; C. a tablet subunit that is provided with a separation mark; or D. (i) a first tablet subunit and (ii) a second capsule subunit that is adhesively joined to said first tablet subunit.

### CROSS-REFERENCE TO RELATED APPLICATION

This application claims the priority of provisional application Ser. No. 60/584,828, filed Jul. 1, 2005.

### FIELD OF THE INVENTION

The invention is concerned with solid pharmaceutical dosage forms that comprise aggregations of preformed subunits that are adhesively joined together.

### BACKGROUND OF THE INVENTION

It is well known to provide dosage forms such as tablets or capsules for handling pre-measured quantities of materials that allow consumers to use various materials without the need to use expensive and cumbersome measuring devices. One reason for the current invention is to allow for flexible dosing of one or more drugs. Japanese unexamined Patent Application Publication H6-9375 by Ito et al (herein, “Ito”) discloses tablets that consist of smaller “unit tablets” (“subunits,” or “tablet subunits,” or “capsule subunits” herein) which are connected by a “cement” that acts as an adhesive. The connecting part is explicitly stated in those cases to be the cement, which is alleged to be breakable while leaving the unit tablets intact. Leaving aside the practicality of breaking through, either mechanically or by dissolving, said cement connecting part, the application discloses production of smaller tablet structure tablets each comprising intact unit tablets. The application fails to disclose tablet subunits that are not “unit tablets.” In addition, the application is specific in the mode of manufacture of said tablets that it discloses. Said mode of manufacture consists of apposing (“mating”) the unit tablets to be joined and then forming a “connecting part” with cement. Said application does not disclose producing said tablets by applying adhesive to an inert subunit that is not simultaneously apposed to or adjacent to another subunit. In addition, because Ito states that the subunits of said application are “unit tablets,” it would not be contemplated to create a score in a unit tablet that is in general use to facilitate tablet breaking. Further, a marking on a tablet, such as a printed mark, that may also facilitate tablet breaking such as by delineating a bisecting part of a tablet subunit, would similarly not be implied by Ito's application. The current invention utilizes the term “separation mark” or “separation marking” to indicate a score, a printed mark, or similar addition to a tablet subunit that may guide or facilitate tablet breaking.

The current invention most clearly differs from Ito's disclosures and the implications of his disclosures in that said application contemplates either that dosage forms of his invention are ingested whole or else are broken or divided only through the “cement” with the tablet subunits considered “building blocks” a la indivisible atoms that form a molecule. In contrast, the current invention discloses dosage forms that are adapted to being broken through a tablet subunit part of said dosage forms. Therefore the methods of breaking said dosage forms of the invention involve breaking through a tablet subunit and not through an adhesive (cement).

The current invention utilizes the general term “inert” to describe a tablet subunit that lacks any active pharmaceutical ingredient (i.e., a “drug”). “Inert” is intended to include tablet subunits that either lack any controlled-release function or that have a controlled release function. The term “inactive” is utilized to indicate a tablet subunit that lacks any active pharmaceutical ingredient and that also lacks any immediate release function. Therefore, any inactive tablet subunit is also considered inert, but an inert tablet subunit may or may not be inactive.

### SUMMARY OF THE INVENTION

The present invention is concerned with novel dosage forms and methods of their manufacture, plus methods of breaking said dosage forms and administration of a broken part of said dosage form.

The invention provides a solid pharmaceutical dosage form comprising a plurality of adhesively-joined subunits. Said dosage form contains at least one or more of the following:
A. (i) a first inert tablet subunit and (ii) a second active subunit;
B. (i) a first tablet subunit with a pharmacologically inactive layer in which said layer has a mass of at least 20 mg and (ii) a second subunit;
C. a separation mark on a tablet subunit; or
D. (i) a first tablet subunit and (ii) a second capsule subunit.

The dosage forms consist of a suitable adhesive substance interposed between and joining (or, “connecting,” or “bonding,”) a plurality of preformed subunits, preferably tablets but possibly involving one or more capsule subunits. The invention differs from the Ito disclosure in that the current invention involves at least one of the following novel aspects:
A. In a preferred embodiment, dosage forms containing at least one tablet subunit with an active drug (“active subunit”) and at least one tablet subunit made of only pharmaceutically inactive material (“inert tablet subunit”) are disclosed herein, whereas Ito only discloses an exemplary placebo tablet made only of inactive subunits;
B. In another preferred embodiment, dosage forms contain two or more active subunits adhesively joined together where at least one of the active subunits has a separation mark that is preferably a score;
C. In another preferred embodiment, dosage forms have two or more active subunits adhesively joined to one or more inert tablet and in said embodiment, a separation mark such as a score is optional;
D. In another embodiment, a capsule subunit may be adhesively joined to a tablet subunit;
E. In another preferred embodiment, the invention involves a dosage form comprising a plurality of active subunits each adjoining an inert subunit that may be readily breakable without damaging any active subunit.
   An inactive tablet subunit of a dosage form of the invention is preferably adapted to be broken, such as a tablet subunit that is provided with a separation mark which may be a score; such a subunit may optionally be an active or an inactive subunit. An active tablet subunit may also be adapted to be conveniently breakable and may be provided with a separation mark such as a score.
   An active subunit herein contains a pharmacologically effective quantity of a drug or drugs. The terms “drug,” “active drug,” “active pharmaceutical ingredient” or “active ingredient,” “active pharmaceutical compound” and the like herein include not only pharmaceuticals such as those that in the United States are regulated by the Food and Drug Administration, but also includes vitamins and minerals.
   In many cases, an inert subunit will link two discrete active subunits. The term “inert tablet subunit” as used herein means a structural unit made on a tabletting apparatus wherein the structural unit contains pharmaceutically acceptable materials, e.g. excipients, diluents, fillers etc. which have no detectable pharmacological effects at the amounts used in the dosage form. When the term “inactive subunit” is used, it is used to describe a subunit which has no detectable pharmacological effects and has no controlled release function.
   The invention provides a method of accurately providing a predetermined part of a dosage form for administration as well as providing a whole dosage form. Preferably but not always, breaking a subunit of the invention will not involve breaking, damaging, dissolving, etc. the adhesive bond between subunits. The invention contemplates that one usage of a dosage form may involve breaking through two or more subunits that also involves breaking through a discrete portion of the adhesive bond that joins them.
   The invention also includes a method of administering a part of an active subunit of a solid pharmaceutical dosage form by breaking said pharmaceutical dosage form through an active subunit to obtain a part comprising an active subunit and then orally administering the part having the active subunit to a patient in need thereof.
   A further embodiment is directed to a method of administering one active subunit of a dosage form having at least two active subunits adhesively joined to an inert tablet subunit by first breaking said inert tablet subunit to form two parts each of which comprises an active subunit and a part of said broken inert subunit and thereafter administering one of said parts to a patient or other appropriate host. The dosage forms and parts formed by breaking are intended to be given enterally, such as orally. Other means of administration, such as through a naso-gastric tube or gastrostomy tube or per rectum, are contemplated as well.
   The invention contemplates crushing a part of a whole dosage form that has been formed by breaking per the methods of the invention, so that administration through a feeding tube and the like may be conveniently accomplished.
   The invention further includes a method of making a solid pharmaceutical dosage form by:
(a) first, applying adhesive to a first active or first inert subunit; and
(b) second, bringing said first active or inert subunit containing adhesive into contact with a second active or inert tablet subunit (that optionally may have adhesive applied as well) and optionally applying pressure to the subunits.
   It will be appreciated that no limitation is placed upon the nature, contents, active or inactive ingredients, or size or shape of either the subunits or the final dosage form, except that the final dosage form should be safe to use and is most preferably able to be taken into the body (e.g., ingested) by its intended user.

The invention utilizes substance(s) with sufficient adhesive ability to allow the subunits to adhere to one another to form one cohesive dosage form. For commercial use, preferably said dosage forms will remain intact through the manufacturing and transport phases until it reaches a patient, nurse, pharmacist, etc. These novel dosage forms have many embodiments and may comprise many different arrangements, many different shapes, types of active ingredient(s), types of inactive ingredient(s), number of subunits, etc. without any limitation. Examples representing embodiments of the invention are given herein to exemplify but not to limit the number of useful possibilities that are within the scope of the invention.

Accordingly it is a primary object of the invention to provide a novel pharmaceutical dosage form which contains one or more active or inactive ingredient(s) in more than one separately-produced active subunit which is adhesively bonded onto an inert linker subunit wherein said dosage form may be separated into two or more parts by breaking said dosage unit at a location or locations within the active subunit to provide a predetermined amount of a drug or drugs contained in said dosage form.

It is also an object of the invention to provide a novel pharmaceutical dosage form which contains one or more active or inactive ingredient(s) in more than one separately-produced active subunit which is adhesively bonded onto an inert subunit wherein said dosage form may be separated into two or more parts by breaking said dosage unit at a location or locations within the inert subunit to provide a predetermined amount of a drug or drugs contained in said dosage form.

It is also an object of the invention to provide a novel pharmaceutical dosage form which contains one or more active or inactive ingredient(s) in more than one separately-produced active subunit which is adhesively bonded onto an inert linker subunit wherein said dosage form may be separated into two or more parts by breaking said dosage unit at locations within one or more of the active subunits and within the inert linker subunit to provide a predetermined amount of a drug or drugs contained in said dosage form.

These and other objects of the invention will become apparent from the present specification.

A “subunit” is a preformed structure classified herein as either as an active subunit, a capsule subunit, or inert tablet subunit or an inactive tablet subunit. Materials such as adhesive substance(s) or film such as hydroxypropyl methylcellulose that may be used to coat a subunit, are not themselves considered subunits.

“Preformed” refers to separate production of a subunit. A tablet subunit of a dosage form of the invention is produced as a tablet, and becomes a subunit when it is part of the dosage form of the invention. Similar considerations apply to a capsule subunit of the invention.

Tablet subunits of the invention may be layered structures as are well known. The invention may encompass a pharmacologically inactive layer of a tablet comprising a plurality of layers to serve as a breaking point and thus said pharmacologically inactive layer may serve a similar function in the invention as does an inert tablet subunit. Preferably said inactive layer has a mass of at least 20 mg and more preferably 50-900 mg; or 150 mg-750 mg or 400-600 mg; a volume of at least 10 cubic mm and more preferably 25 cubic mm; and/or a length along the longest axis of the dosage form of at least 1 mm, and more preferably 2 mm. Thus said inactive layer may play a role in dosage form subdivision by serving as a breaking region.

“ “Tablet” and “capsule” are defined in their usual ways. Active subunits may contain one or more drugs.

“Pharmaceutical dosage form” herein refers to a solid dosage form containing two or more subunits adhesively bonded together. The preferred solid dosage form is an oral dosage form.

### BRIEF DESCRIPTION OF THE DRAWINGS

All drawings depict dosage forms of the invention in which contiguous subunits are adhesively joined. All views are external views.

FIG. 1 is a drawing of a top view of the dosage form of the invention depicting three subunits.

FIG. 2 is a schematic top view of a dime-shaped dosage form that has three subunits, one of which is scored.

FIG. 3 is a schematic top view depicting four active subunits each joined by an adhesive substance to an inert tablet subunit.

FIG. 4 depicts five tablet subunits, two of which each adhesively join three other subunits.

FIG. 5 is a schematic top view that depicts a dosage form of the present invention in the shape of a capsule. The inert tablet subunit has two indentations that each contain a preformed active subunits.

FIG. 6 is a schematic side view depicting a dosage form Consisting of three scored, adhesively joined active tablet subunits.

FIG. 7 is a schematic side view depicting a dosage form consisting of three active tablet subunits each of which is adhesively joined to two inert tablet subunits.

FIG. 8 is a schematic top view of a three-subunit dosage form.

FIG. 9 is a schematic top view of a four-active subunit dosage form in which the four active subunits are adhesively bonded together.

FIG. 10 depicts a side view of a dosage form containing four tablet subunits.

FIG. 11 depicts a dosage form with five subunits.

FIG. 12 depicts a dosage form with three subunits joined together adhesively.

FIG. 13 depicts a dosage form with three tablet subunits, none of which are scored.

FIG. 14 depicts a dosage form with three subunits, one of which is scored.

FIG. 15 is an exploded depiction of a two-subunit dosage form, one of which is a trilayer tablet subunit.

FIG. 16 is a partially exploded perspective view of a dosage form having two active subunits, one of which is a tablet subunit and one of which is a capsule subunit, and an inert tablet subunit.

FIG. 17 is a perspective view of a partially exploded dosage form having four active subunits and an inert tablet subunit adjoining said active subunits.

FIG. 18 is a perspective view of a partially exploded dosage form having two active subunits and an inert tablet subunit with score marks placed in the inert tablet subunit.

### DETAILED DESCRIPTION OF THE INVENTION

The invention contemplates that an active subunit comprise a drug or drugs, that a capsule subunit is an active subunit (excepting placebo formulations for clinical trials and the like), and that inert tablet subunits lack a drug.

The dosage forms of the invention may be made by joining individual tablets or capsules which are shaped in any desired configuration by such means as the use of tablet punches and dies (tablets), or encapsulating equipment (capsules). Said methods of manufacture are not limiting. The method of joining involves material(s) with adhesive properties. There may be additionally be materials with non-adhesive characteristics between the subunits.

The dosage forms may comprise active subunits and inert tablet subunits having a plurality of various cross-sectional shapes, including without limitation round tablets, half-round, quarter-round, oval, trapezoidal, triangular, rectangular, etc., that are be adhesively bonded to each other. In a preferred embodiment of the invention, the active subunits and/or capsule subunits may be separated from each other in a convenient manner by an end-user, nurse, pharmacist, etc. without damaging the function of said subunits. It is preferred that such non-damaging separation of at least one active subunit (with or without a potion of the inert tablet subunit) from the dosage form will be able to be performed manually; however, there may be cases in which manual separation is inconvenient or not possible without damaging one or more subunits. In such cases, it is within the scope of the invention that convenient mechanical means of separation of at least one subunit from the entire dosage form may exist, utilizing such implements as a commercially-available tablet cutter, kitchen knife, etc. It is also within the scope of the invention that because the invention contemplates dosage forms comprising subunits with a variety of shapes, with some dosage forms containing more than two subunits, it may not be convenient to use a standard tablet cutter, and it may therefore be desirable to create a tablet cutter specifically adapted for the specific dosage form to optimize ease of non-damaging subunit separation.

By use of the procedures of the invention, pharmaceutical dosage forms containing two or more separate subunits may be made.

In general, an effective amount of an adhesive is used to join any two surfaces together. No limitation is intended as to the amount of adhesive needed. The amount of adhesive needed is dependent on multiple factors, such as the size and density of the dosage form. In general, a minimum amount of adhesive is preferable. Generally, the amount of adhesive may comprise from 0.1 to 5 mils of adhesive spread over an area of approximately 0.5 to 100 sq mm., more or less, depending on the particular tablet size and area and number of the surfaces being joined together. In the case of veterinary dosage forms, such areas may be increased significantly.

FIG. 1 is a schematic side view of the invention that depicts two active subunits, 52 and 54, respectively, joined by inert tablet subunit 56. Adhesive joins active subunit 52 to one side of the inert tablet subunit 56 and also joins active subunit 54 to the other side of the inert tablet subunit 56. Active subunits 52 and 54 are conveniently separable from each other by, for example, cutting through the inert tablet subunit 56, or by grasping active subunits 52 and 54 and applying force centered on the inert tablet subunit 56, including rotary force or perpendicular force. Active subunits 52 and 54 are substantially identical.

The figures are not necessarily drawn to scale and are not intended to limit the size or shape of the dosage form or any subunit. Inert tablet subunits such as subunit 56 in Fig. generally may have a mass greater than 20 mg or more preferably form 50 mg to 900 mg, or even more preferably from 150-600 mg or even more preferably form 400-500 mg. The length and thickness will be selected ensure that the dosage form may be enterally administrable while being sufficiently robust to avoid undue amounts of breakage during packing, shipping and handling.

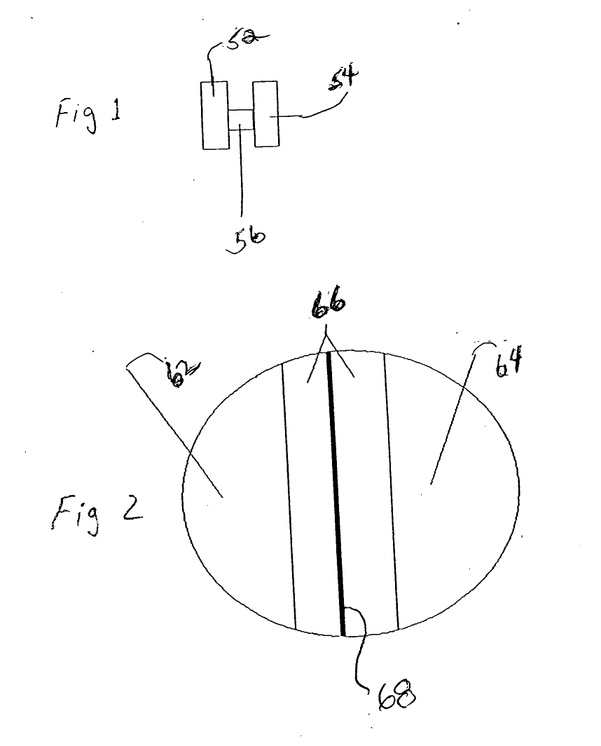

As shown in FIG. 2, a dosage form that is circular (coin-shaped such as dime-shaped) is shown on top view having active tablet subunits 62 and 6, that are each joined to a scored inert tablet subunit 66 with score 68. Inert tablet subunit 66 may be conveniently manually or mechanically broken without damaging either active subunit 62 or active subunit 64. Optionally a dosage form of this form could have one or both active tablets scored as well.

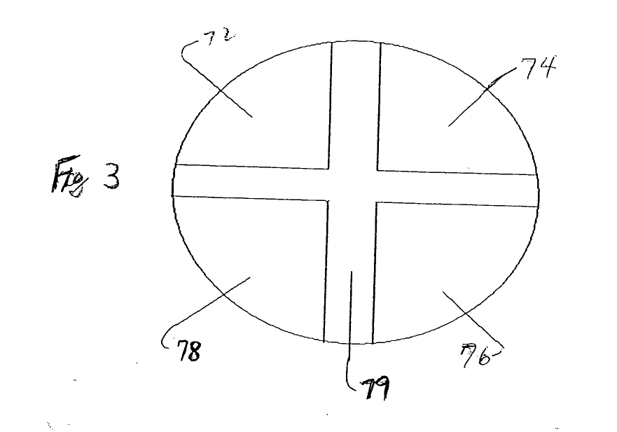

FIG. 3 depicts four active subunits, 72, 74, 76 and 78 joined via an adhesive substance to an inert tablet subunit element 79 that if broken can provide one or more subunits to be ingested.

FIG. 4 is a drawing which shows three active subunits, 80, 82 and 84, none of which are connected to each other, but all of which are connected to inert tablet subunits 85 and 86 on opposite ends of the active subunits. Breaking the dosage form through the inert subunits may be accomplished without damaging any active subunit. Not shown is a similar design in which the active subunits are attached only on one end to an inert tablet subunit. In practice, the gaps 87 and 89, between the active subunits, may be smaller than shown. FIG. 4 does not exclude the possibility that the active subunits so arranged may actually touch.

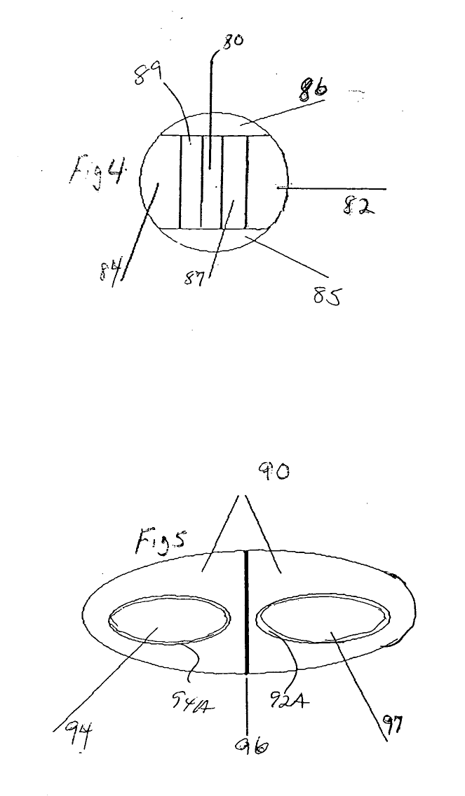

FIG. 5 is a drawing of a dosage form comprising three subunits. Active subunits 92 and 94 are adhesively bonded to indentations (depressions) 92A and 94A which are formed in subunit 90. Subunit 90 could be a active subunit containing an active ingredient(s) or subunit 90 could also be an inert tablet subunit. Dosage forms similar to FIG. 5 may be made in a variety of shapes with varying shaped indentations or depressions. The entire dosage form is ingestible. The dosage form may be broken at score 96 to separate the active subunits in the dosage form into two parts.

As shown in FIG. 6, a dosage form of the invention may comprise a stacked multilayer arrangement of substantially flat active subunits 8, 10 and 12 that contain, respectively, score marks 5, 7, and 9 and which are adhesively bonded together with small quantities of adhesive 6 to form a layered structure.

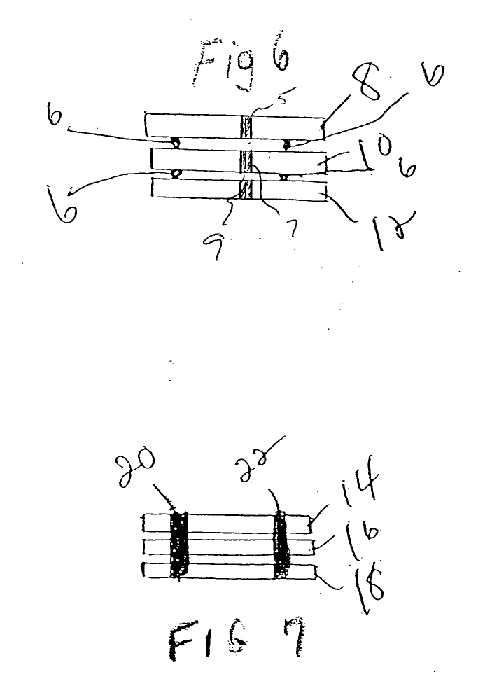

FIG. 7 shows a dosage form in which three active subunits, 14, 16, and 18 are held together with inert tablet subunits 20 and 22 that are adhesively joined to a side of each of the active subunits. While FIG. 7 shows two inert tablet subunits on the side of the tablets, it is possible to use only one, two, or a plurality of inert tablet subunits that may be positioned to form a stable and useful dosage form.

FIG. 8 depicts a top view of a dosage form having an inert tablet subunit 34 that joins ends 36 and 38 which contain active ingredients. Lines 40 and 42 represent the adhesively joined edges of the active subunits. If desired, it is convenient to break through the inert tablet subunit without affecting either active subunit.

FIG. 9 shows a top view of a four-active subunit dosage form that is made by adhesively bonding together segments 24, 26, 28 and 30, which each contribute 90° of arc to the final shape of the dosage form. This dosage form is particularly useful when it is desirable to simultaneously administer several different active drugs. Score 27 approximately bisects subunit 30 and score 29 approximately bisects subunit 26. The dosage form is therefore conveniently divisible through both scores, that form a continuous linear indentation across the dosage form. Adhesive 6 that is the same type of adhesive used in FIG. 6 joins the subunits together.

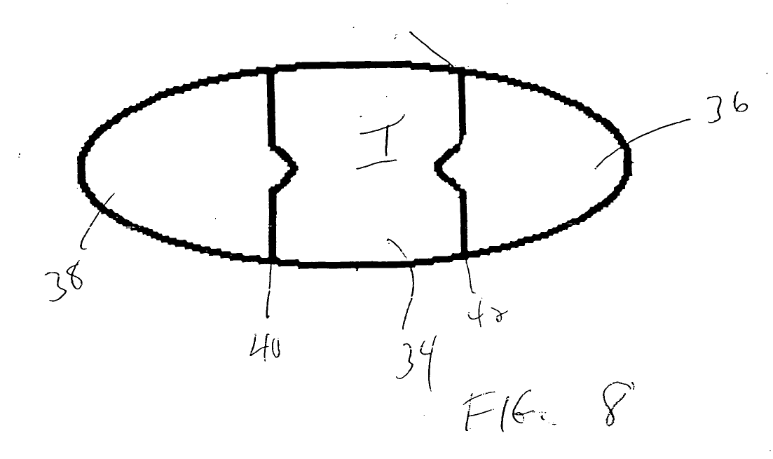

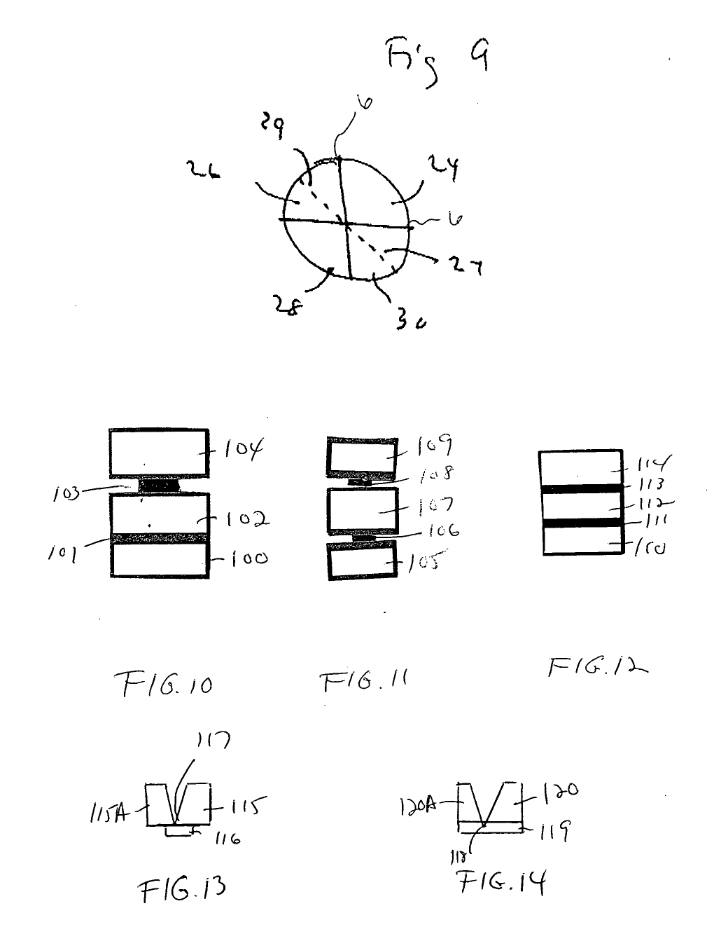

FIG. 10 shows a drawing of a three-active subunit dosage form in which an outer active subunit 100 is bonded with adhesive layer 101 to a middle active subunit 102. Outer subunit 104 is bonded with adhesive to inert tablet subunit 103, which is adhesively bonded to middle active subunit 102.

FIG. 11 shows a drawing of a three-active subunit dosage form in which the outer active subunits 105 and 109 are each adhesively bonded to inert tablet subunits 106 and 108, respectively, said inert tablet subunits being adhesively bonded to middle active subunit 107.

FIG. 12 shows a drawing of a three-active subunit dosage form in which active subunit 110 is bonded with adhesive 111 to middle active subunit 112. Active subunit 114 is bonded with adhesive 113 to middle active subunit 112.

FIG. 13 shows inert tablet subunit 116 adhesively bonded to active subunits 115 and 115A. The active subunits contain the same drug. The dosage form may be broken conveniently between subunits 115 and 115A through subunit 116.

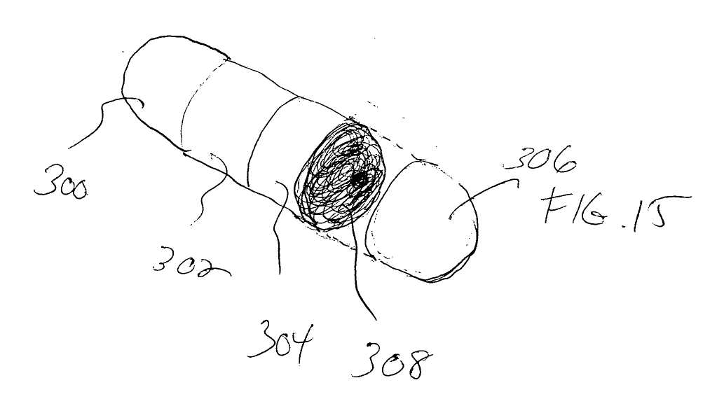

FIG. 14 depicts inert tablet subunit 118 with a score that is between different active subunits 120 and 120A, facilitating tablet breaking when desired.

FIG. 15 depicts in an external view a dosage form consisting of two tablet subunits adhesively joined together at interface 308 that comprises an adhesive substance. The tablet subunit that consists of layers 300, 302 and 304 is produced separately on a tri-layer tablet press. The vertical lines in between layers 300 and 302, and between layers 302 and 304, represent the interfaces between the layers that are all of different colors. Layer 300 contains a therapeutic quantity of amlodipine besylate (amlodipine) and layer 304 contains a therapeutic quantity of chlorthalidone. Layer 302 is formed from an inactive granulation and, due to mixing between layers in the tablet-forming process, contains pharmacologically ineffective quantities of both amlodipine and chlorthalidone. Said trilayer tablet that is a subunit of the dosage form depicted in FIG. 15 is produced first by allowing a granulation containing amlodipine and suitable excipients into the die, then tamping; then allowing a granulation lacking an active ingredient into the die, then tamping, then allowing a granulation containing amlodipine into the die, then pre-compressing and then fully compressing to form a coherent tablet. The upper punch is beveled and the lower punch is flat-faced.

Tablet subunit 306 contains benazepril and is produced separately in a conventional fashion. Subunit 306 is adhesively joined to the tri-layer tablet described above with shellac.

An advantage of the dosage form depicted in FIG. 15 is that a tri-layer tablet such as is described above (layers 300,302 and 304) may be used to produce the more complex, three-active drug, dosage form of FIG. 15 while retaining the ability to break the dosage form through layer 302 and producing two useful dosage forms. One dosage form would contain a therapeutic quantity of amlodipine only and the other would only contain therapeutic quantities of chlorthalidone and benazepril only.

Numerous other variants of the design of the dosage form depicted in FIG. 15 may be utilized within the present invention.

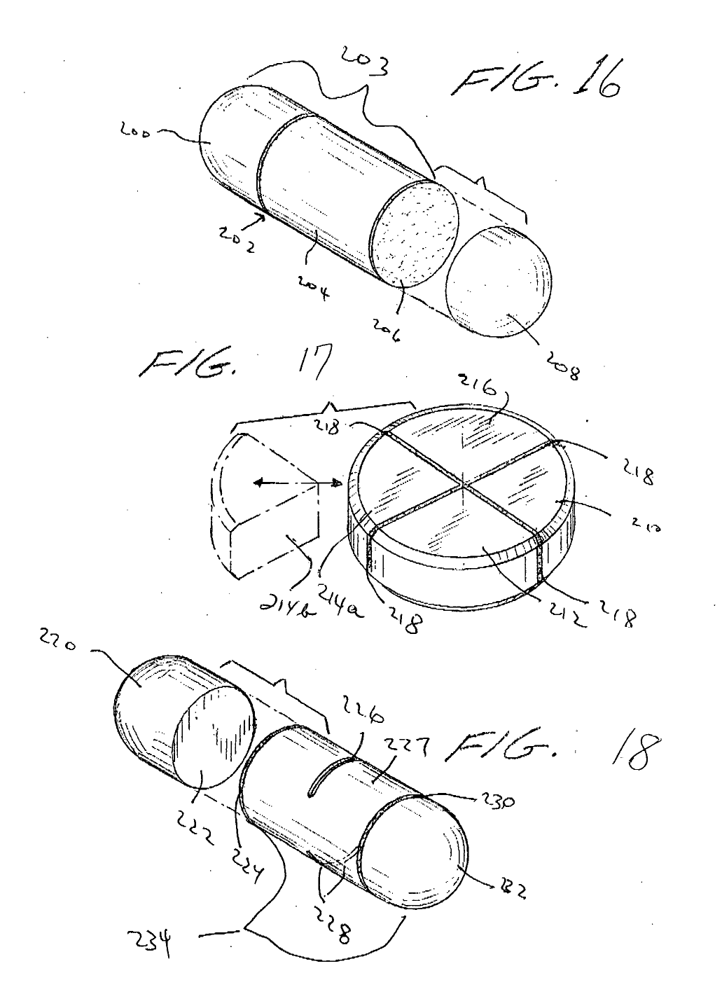

FIG. 16 is a partially exploded perspective view of a dosage form that consists of part 203 adhesively joined to subunit 208. Subunit 200 in this example is a capsule subunit. Part 203 contains subunit 200 and inert tablet subunit 204.

The length of subunit 204 relative to the other subunits makes it relatively easy to break through.

FIG. 17 depicts an external view of a dosage form that contains four active tablet subunits that are all adhesively joined to linker tablet subunit 218 that is inactive. Subunits 210, 212, 214, and 216 are all active. In the current example, each of said active subunits contains a therapeutically effective quantity of a different drug. In alternative embodiments, two or more active subunits may contain the identical drug.

Note that tablet subunit 214a is shown in its proper position in the dosage form of FIG. 17 and also is shown in a phantom exploded view as 214b.

FIG. 18 depicts a partially exploded external view of a dosage form containing part 234 comprising a double-scored inactive tablet subunit 227, interface 230, tablet subunit 232, and shellac layer 224; and tablet subunit 220. Top score 226 and bottom score 228 are created manually with a file.

Scores 226 and 228 adapt tablet subunit 227 to be broken more easily than if no scores were present. Optionally, subunit 227 could have been a scored active tablet subunit.

The tablet subunits that are adhesively bonded together may be made using such techniques as are employed in the pharmaceutical industry to produce conventional tablets. These techniques include, without limitation, wet granulation methods; dry granulation techniques, such as slugging and grinding; or direct compression powder blends.

Pharmaceutical Uses

Generally, many useful solid dosage forms may be usefully produced by the methods of the invention. There are several practical advantages to the invention. These include, without limitation:

A. There will no longer be a need to co-formulate different products when desiring to combine them into one dosage form. Not only will the methods of the invention save time and money in such development, but such savings will make it easier to initiate testing of combination therapies. The vexing issue of incompatibilities between different formulations will cease to be limiting. Several other physical and chemical considerations will no longer be limiting as well.
B. Two or more doses of the same drug product may be produced separately, then joined as tablet subunits or capsule subunits. If accurately separable from each other, more precise dosing will then be available than is currently achievable with conventional scored tablets. Regarding joining two capsules, there is currently no marketed product in the United States that provides the ability to separate a capsule dosage form into two capsule subunits.
C. The invention allows a tablet and a capsule to be joined together in one dosage form, which advances the pharmaceutical art in a novel and potentially important way.
D. Physician and patient acceptance of combination therapies, as well as acceptance by academics, government, regulatory bodies, and society at large may be enhanced by allowing flexibility in dosing of combination products by following the methods of the invention.
E. The invention is well-suited for formulating combination drug products, i.e. dosage forms which contain two more active ingredients. When the dosage forms of the invention comprise more than one active drug, the dosage forms of the invention may provide a convenient manner of improving patient compliance with dosing regimens while at the same time minimizing formulation problems. These benefits are in addition to the advantage of being able to discontinue therapy with one or more drugs by removal of one or more subunits containing a particular drug from the dosage form. A further benefit is the simplification of the prescribing and dispensing of multiple individual drug products.
F. Patients allergic to one member of a combination product can break some preferred embodiments of the invention and ingest one drug of the combination tablet, something not able to be safely done currently.
   The invention provides a method of making a dosage form of a combination of different drugs where the different drugs may be separated from the combination without any trace amount of another drug being present in combination with another drug. For example, when multilayer tablets are formed on a multilayer tablet press, it is difficult or nearly impossible to exclude all traces of the granulation for one layer from appearing in a compressed layer of a another granulation due to air currents and vibration which cause the granulations to disperse or form aerosol like dispersions in the area where the granulations are fed into the dies on the multilayer press. The present invention allows the separate components of the combination dosage form to be separately tabletted and adhesively joined in such a manner that there is no cross-migration of the individual drugs between the separate active units in a dosage form according to the present invention. Thus when the active subunits are obtained apart from the dosage form of the invention, there is no cross-contamination of the individual drugs. This is particular important when patients are allergic to a particular drug.
   As mentioned above, the invention contemplates that solid dosage forms may generally benefit from the invention's teachings. What follows are some specific examples of products that either may be combined together Some useful combinations of drugs are, with examples in parentheses:
   Combinations of anti-anginal agents. These include any combination of the following drug classes:
A. Calcium channel blocking agents (amlodipine salt, diltiazem, lercanidipine)
B. Beta-blocker (atenolol, metoprolol, propranolol)
C. Organic nitrate preparation (isosorbide mononitrate or dinitrate);
   Combination of anti-anginal agent (see above) with anti-platelet agent, such as aspirin or clopidogrel;
   Combination of any two hypoglycemic agents;
   Combination of potassium salt (preferably KCl) and any thiazide-type or loop diuretic;
   Combination of hypolipidemic agent with any of a variety of representatives of drug classes: hypoglycemic agent, anti-platelet agent, anti-anginal agent, organic nitrate, antihypertensive agent:
Hypolipidemic agent may be a:
Statin (simvastatin, atorvastatin with or without torcetrarib, rosuvastatin, lovastatin, rosuvastatin, fluvastatin), a fibrate (fenofibrate, gemfibrozil, bezafibrate, ciprofibrate, clofibrate), or member of other classes (niacin, ezetimide, acipimox)
   Hypoglycemic agents:
Thiazolidinediones: Pioglitazone, rosiglitazone Sulfonylureas: Glyburide, glipizide, glimepiride, chlorpropamide
Biguanides: Metformin
Meglitinides: Nateglinide, repaglinide
Glucosidase inhibitors: Acarbose, miglitol;
   A member from two or more of the following three classes:
A. Diuretic (HCTZ, furosemide)
B. Digoxin (and other cardiac glycosides)
C. Beta-blocker approved for congestive heart failure treatment (metoprolol, carvedilol);
   Combinations of two or more antihypertensive agents, most preferably one member from different classes as described below:
   Beta-blockers:
Acebutolol, atenolol, bisoprolol, celiprolol, metoprolol, mebivolol, carvedilol (a mixed alpha-beta blocker), nadolol, oxprenolol, penbutolol, pindolol, propranolol, timolol, betaxolol, carteolol,
   Calcium antagonists (calcium-channel blockers):
Nifedipine, amlodipine, verapamil, diltiazem, nisoldipine, felodipine, isradipine, lacidipine, lercanidipine, nicardipine, manidipine
   Thiazide-type diuretics (with or without potassium-retaining diuretics such as triamterene, amiloride, or spironolactone:
Hydrochlorothiazide, chlorothiazide, cyclopenthiazide, polythiazide, bendrofluazide, hydroflumethiazide, chlorthalidone, indapamide, methylclothiazide, metolazone
   Angiotensin converting enzyme inhibitors:
Captopril, enalapril, lisinopril, ramipril, trandolapril, quinapril, perindopril, moexipril, benazepril, fosinopril
   Angiotensin receptor blockers:
Losartan, valsartan, candesartan, telmisartan, eprosartan, irbesartan
   High-ceiling (loop) diuretics (with or without potassium-retaining diuretics such as triamterene, amiloride, or spironolactone):
Furosemide, torsemide, ethacrynic acid, bumetamide
   Aldosterone antagonist diuretics:
Spironolactone, eplerenone
   Alpha-blockers:
Doxazosin, terazosin, prazosin, indoramin, labetolol (a mixed alpha-beta blocker)
   Central alpha-agonists:
Clonidine, methyldopa
   Imidazoline:
Moxonidine
   Direct vasodilators:
Hydralazine, minoxidil
Adrenergic neuronal blocker:
Guanethidine.
   It will be appreciated that certain combinations, such as a beta-blocker and a diuretic, are more preferred than others, such as verapamil and a beta-blocker, or furosemide and hydrochlorothiazide. The same is true for other combinations.
   In addition to the above, many products benefit from accurate separability, such as into half-doses, quarter-doses, etc. Some prominent examples that may benefit more than most include narrow therapeutic index drugs. Examples of these are warfarin sodium and other coumarins, L-thyroxine, and digoxin. Other examples especially benefiting from being formulated as to be accurately separated when desired are vasoactive drugs such as calcium antagonists and beta-blockers. For example, many if not all of the drugs listed above regarding combinations may be usefully formulated per the invention and could then be the only active drug in a dosage form of the invention. Thus, a great many single drug products can be precisely separated one from the other by the invention; also, a combination drug product can be placed together in a subunit and thus be precisely separated as a fixed dose combination,
   No limitation is intended regarding the number of subunits in the above or any of the instructive examples provided. No limitation is intended as to the many examples of useful combinations of dosage subunits that may benefit from this invention. Numerous other monoagents, in addition to those listed above may be formulated alone, or in combination with other drugs using the procedures of the invention.
   Manufacturing

Preferred adhesive substances include, without limitation, the following:

| MFG. | Material |
|---|---|
| Rohm GmbH & Co. KG | Methacrylic Acid Copolymer |
| | Type B NF |
| | (Eudragit ® S 100) |
| Rohm GmbH & Co. KG | Methacrylic Acid Copolymer |
| | Type C NF |
| | (Eudragit ® L 100-55) |
| Rohm GmbH & Co. KG | Methacrylic Acid Copolymer |
| | Type A NF |
| | (Eudragit ® L 100) |
| Mantrose-Haeuser Company | #4 Pharmaceutical Glaze |
| | (Shellac) |
| | 45/200 |
| Mantrose-Haeuser Company | #4 Pharmaceutical Glaze |
| | (Shellac) |
| | 45/200 with Stabilized |
| | Shellac |

In Addition, Suitable Adhesive Materials May Be Selected From the Following List:
As synthetic adhesives, one may cite the following specific examples.
As thermosetting resin adhesives, the chemical reaction types of urea resin, melamine resin, phenol resin, epoxy resin, polyester resin, polyimide resin, and the like.
As thermoplastic resin adhesives, the solvent vaporization types of solvent-type vinyl acetate resin, emulsion-type vinyl acetate resin, vinyl chloride-vinyl acetate copolymer resin, nitrocellulose, acryl-vinyl acetate copolymer resin, ethylene-vinyl acetate copolymer resin, and the like; the chemical reaction types of cyanoacrylate, anaerobic acrylic resin, urethane resin, and the like; the cooling (hot-melt) types of ethylene-vinyl acetate copolymer resin, polyamide, polyester, and the like; the pressure sensitive type of acrylic resin, and the like.
As synthetic rubber adhesives, the solvent-vaporization types of chloroprene rubber, nitrile rubber, reclaimed rubber, latex, and the like; the chemical-reaction type of urethane rubber, and the like; the cooling (hot-melt) type of polystyrene-polyisoprene-polystyrene block copolymer, and the like; the pressure-sensitive types of butyl rubber, polyisobutylene rubber, silicone, and the like.
As polymer alloy adhesives, the chemical-reaction types of vinyl-phenolic, rubber-phenolic, epoxy-phenolic, nylon-epoxy, nitrile-epoxy, and the like.
   As natural adhesives, one may cite the following specific examples.
The solvent-vaporization types of starch, casein, dextrin, gum Arabic, and the like; the cooling (hot-melt) types of asphalt, glue, rosin, and the like; and the pressure-sensitive types of conjugated polysaccharides, natural rubber, and the like.
   As adhesives, one may cite the following specific examples.
As rubber adhesives, mixtures of tackifiers, softeners, antioxidants, and the like with elastomer.
As acrylic adhesives, synthetic products of ester acrylate, and the like.
As silicone adhesives, the substances produced from silicone rubber and silicone resin of the aqueous-emulsion type, oligomer type, and hot-melt type.
Otherwise, polyether adhesive, polyurethane adhesive, etc. Furthermore, it is possible to process these adhesives in tape and sheet form for use.
   As viscous substances producing viscosity by water, one may cite the following specific examples.
As polymers possessing carboxylic groups, acrylic acid copolymers and the like with acrylic acid as the constituent monomers; and as substances with salt there are monovalent and bivalent salts and the like of sodium salt, potassium salt, magnesium salt, and calcium salt.
As cellulose ethers, carboxymethyl cellulose sodium, hydroxypropyl cellulose, hydroxypropylmethyl cellulose, methyl cellulose, crystal cellulose-carboxymethyl cellulose sodium, and the like.
As natural viscous substances, mucin, agar, gelatin, pectin, carrageenin, sodium alginate, locust bean gum, chitin, chitosan, traganth powder, and the like.
Polyethylene glycol with a molecular weight of 200,000 or more, and the like.
   As viscous substances that produce viscosity by organic solvents such as alcohol and the like, one may cite the following specific examples.
Hydroxypropyl cellulose, hydroxypropylmethyl cellulose, ethyl cellulose, hydroxypropylmethyl cellulose phthalate, hydroxymethyl cellulose acetate-succinate, acrylic acid copolymer, shellac, waxes, and the like.
   Active Formulas
   Direct Compression Formulas

The following formulations are used in making single layer tablets that are subsequently adhesively joined. A Manesty 16 station Beta Press (single layer rotary tablet press) is used to make the amlodipine and benazepril tablets. The two formulations are directly compressible powder blends. The blending both of the amlodipine formulation and the benazepril formulation is performed in a Patterson-Kelly “V” blender. The tablets are compressed using ¼ inch flat faced beveled edge tablet punches to a hardness of 25 kiloponds. The tablet weight is 62.0 mg for the amlodipine tablet and 54.0 mg for the benazepril tablet. Weights in mg of the granulation comprising each segment follow:

| | Mg. |
|---|---|
| Amlodipine Tablet Formula | |
| Dibasic calcium phosphate anhydrous | 51.13 |
| Amlodipine besylate | 7.15 |
| Sodium starch glycolate (Explotab ®) | 2.48 |
| Magnesium stearate | 0.93 |
| FD&C Blue #1 Aluminum Lake | 0.31 |
| Total | 62.00 |
| Benazepril Tablet Formula | |
| Lactose 310 monohydrate | 42.03 |
| Benazepril HCl | 9.00 |
| Crospovidone | 2.16 |
| Magnesium stearate | 0.54 |
| FD&C Red #40 Aluminum Lake | 0.27 |
| Total | 54.00 |

Fluid Bed Wet Granulation Formulations

The following two formulas are used to manufacture wet granulations which are subsequently compressed into single layer tablets and may be adhesively joined together.

The wet granulations are all made using the following fluid bed granulator procedure.
1. Screen the Active Pharmaceutical Ingredient (API) (amlodipine or chlorthalidone), Starch 1500, Sodium Starch Glycolate and Microcrystalline Cellulose through a US 20 mesh sieve.
2. Screen the Magnesium Stearate through a US 30-mesh sieve.
3. Weigh all of the ingredients. Balance.
4. Prepare 12% Starch slurry by adding the 150 g of Starch 1500 to the 11.00 g of Water while stirring with a Lightnin mixer with a propeller shaft. Mix for a minimum of 10 minutes. Mix continuously during the granulation procedure.
5. Place the ½ Microcrystalline Cellulose, API, 22.5 g of Sodium Starch Glycolate, and the remaining Microcrystalline Cellulose into a Mendel Fluidbed Dryer/Granulator set up with the 20 liter bowl and a top spray nozzle. Mix for a minimum of 10 minutes before starting to spray. Set up parameters are:
   Inlet Temp: 60 to 70° C.
   Spray Rate: 25 to 35 g/min
   Atomization Pressure: 1 bar
6. Spray all of the Starch 1500 slurry on to the blend.
7. Dry the granulation to about 2% moisture.
8. Mill the dried granulation using the Comil with a 20 mesh equivalent screen.
9. Place the milled granulation into a 4 quart V-Blender and add the remaining 12.5 g of Sodium Starch Glycolate and mix for 5 minutes.
10. Add the Magnesium Stearate to the V-Blender and mix for 3 minutes.

The tablets are compressed on a Manesty 16 station Beta Press, single layer rotary tablet press. The tablets are compressed using ¼ inch flat faced beveled edge tablet punches to a hardness of 20-25 kiloponds. The amlodipine tablet weight is 60.0 mg and the chlorthalidone tablet weights 70 mg.

The following give formulae for use in producing granulations that may be used in tablets of the invention.

| | | | Amount |
|---|---|---|---|
| | % of Wet | % per | per |
| Ingredient | Granulation | tablet | tablet |
| Amlodipine besylate | 17.138 | 16.667 | 10 |
| Calcium Phosphate, | 54.842 | 53.333 | 32 |
| anhydrous | | | |
| Starch 1500 | 15.424 | 15.000 | 9 |
| Avicel PH102 | 10.282 | 10.000 | 6 |
| Sodium Starch | 2.314 | 2.250 | 1.35 |
| Glycolate | | | |
| (Intragranular) | | | |
| Sodium Starch | — | 2.250 | 1.35 |
| Glycolate | | | |
| (Extragranular) | | | |
| Magnesium Stearate | — | 0.500 | 0.3 |
| Water (for 12% | As Req'd | — | — |
| Starch Slurry) | | | |
| Total | 100% | 100% | 60 mg |

| | | | Amount |
|---|---|---|---|
| | % of Wet | % per | per |
| Ingredient | Granulation | tablet | tablet |
| Chlorthalidone | 36.724 | 35.714 | 25 |
| Calcium Phosphate, | 35.255 | 34.286 | 24 |
| anhydrous | | | |
| Starch 1500 | 15.424 | 15.000 | 10.5 |
| Avicel PH102 | 10.283 | 10.000 | 7 |
| Sodium Starch | 2.314 | 2.250 | 1.575 |
| Glycolate | | | |
| (Intragranular) | | | |
| Sodium Starch | — | 2.250 | 1.575 |
| Glycolate | | | |
| (Extragranular) | | | |
| Magnesium Stearate | — | 0.500 | 0.350 |
| Water (for 12% | As Req'd | — | — |
| Starch Slurry) | | | |
| Total | 100% | 100% | 70 mg |

Inactive Formulas
Direct Compression Formulas

The following formulations are used in making single layer tablets that may be subsequently adhesively joined to other tablets. A Manesty 16 station Beta Press (single layer rotary tablet press) is used to make the tablets. The three formulations are directly compressible powder blends. The blending is performed in a Patterson-Kelly “V” blender. The tablets are compressed using ¼ inch flat faced beveled edge tablet punches to a hardness of 20-25 kiloponds. Weights in mg of the granulation comprising each tablet follow:

| | Mg. |
|---|---|
| Tablet 1 | |
| Nu-Tab ® (Compressible sugar 30/35 N.F.) | 194.00 |
| Tablet 2 | |
| Dibasic calcium phosphate anhydrous | 158.59 |
| Magnesium stearate | 2.79 |
| PVP K-30 | 2.62 |
| Total | 164.00 |
| Tablet 3 | |
| Lactose 316 Fast Flo | 70.00 |
| Microcrystalline cellulose (Avicel | 24.00 |
| PH102 ®) | |
| Crospovidone | 4.00 |
| Colloidal silicon dioxide | 0.50 |
| Hydrogenated vegetable oil | 1.00 |
| FD&C Red #40 Aluminum Lake | 0.50 |

Fluid Bed Wet Granulation Formulation

The following formula is used to manufacture a wet inactive granulation which is subsequently compressed into a single layer tablet. The wet granulation is made using the following fluid bed granulator procedure.
11. Screen the Starch 1500, Sodium Starch Glycolate and Microcrystalline Cellulose through a US 20 mesh sieve.
12. Screen the Magnesium Stearate through a US 30-mesh sieve.
13. Weigh all of the ingredients. Balance.
14. Prepare 12% Starch slurry by adding the 150 g of Starch 1500 to the 1100 g of Water while stirring with a Lightnin mixer with a propeller shaft. Mix for a minimum of 10 minutes. Mix continuously during the granulation procedure.
15. Place the ½ Microcrystalline Cellulose, 22.5 g of Sodium Starch Glycolate, and the remaining Microcrystalline Cellulose into a Mendel Fluidbed Dryer/Granulator set up with the 20 liter bowl and a top spray nozzle. Mix for a minimum of 10 minutes before starting to spray. Set up parameters are:
   Inlet Temp: 60 to 70° C.
   Spray Rate: 25 to 35 g/min
   Atomization Pressure: 1 bar
16. Spray all of the Starch 1500 slurry on to the blend.
17. Dry the granulation to about 2% moisture.
18. Mill the dried granulation using the Comil with a 20 mesh equivalent screen.
19. Place the milled granulation into a 4 quart V-Blender and add the remaining 12.5 g of Sodium Starch Glycolate and mix for 5 minutes.
20. Add the Magnesium Stearate to the V-Blender and mix for 3 minutes.

The tablets are compressed on a Manesty 16 station Beta Press, single layer rotary tablet press. The tablets are compressed using ¼ inch flat faced beveled edge tablet punches to a hardness of 20-25 kiloponds. The tablet weight is 70.0 mg.

The following formula may be used to produce a granulation that may produce tablet subunits of the invention.

Inactive Wet Granulation

| | | | Amount |
|---|---|---|---|
| | Percent per | Amount per | per Batch |
| Ingredient | Tablet | Tablet (mg) | (g) |
| Starch 1500 | 15.00% | 10.5 | 150 |
| (Colorcon) | | | |
| Microcrystalline | 80.00% | 56.0 | 800 |
| Cellulose (Avicel | | | |
| PH102) (FMC) | | | |
| Sodium Starch | 4.5% | 3.15 | 45 |
| Glycolate | | | |
| Magnesium | 0.5% | 0.35 | 5 |
| Stearate. | | | |
| (Mallinckrodt) | | | |
| Water | | | 1100* |
| (not part of final | | | |
| blend) | | | |
| | 100% | 70 | 1000 |

The above active and inactive formulas are also suitable for capsule filling. The formulas have to be adjusted to accommodate the desired capsule fill volume.
To make an adhesively bonded pharmaceutical dosage form, a tablet may be made using one of the formulas above to compress an amlodipine tablet using techniques well known in the art of tablet manufacture. An inert tablet could be made using one of the above formulas. One of the above formulations may be used to manufacture a chlorthalidone tablet according to standard techniques. Adhesive, such as, Mantrose-Heauser #4 Pharmaceutical Glaze (shellac) 45/200 is applied to both mating sides of the inert tablet. The mating side of the amlodipine tablet is joined to one of the inert tablet mating sides and the chlorthalidone tablet (mating side) to the other. Adhesive may or may not be applied to the mating sides of the active tablets prior to mating to the inert tablet. The tablets are allowed to dry with or without pressure applied under ambient conditions or in a drying tunnel.
Numerous other means of creating the dosage forms of the invention may be produced using the formulas given above to allow production of tablets or capsules. An almost infinite number of dosage forms may be produced according to the invention using any of the many tablet and capsule formulas that are known to those skilled in the art and that will be, but have not yet been, developed.
Shellac solutions, preferably ranging from 29% w/w to 36% w/w solids in a solvent comprising ethanol may be used as an adhesive. An applicator is dipped into thoroughly-mixed shellac and the shellac solution is applied to the joining surfaces. A tablet subunit is joined to a tablet subunit(s); a capsule subunit is joined to a capsule subunit(s); tablet subunits may be joined to a capsule subunit(s) and tablet subunits or capsule subunits are joined with linkers between them. No limitation to the number of subunits or linkers is intended.
Examples of Methods of Joining the Subunits of the Dosage Form
1. About one ml. of the shellac solution is applied to the entire joining surface of a tablet subunit. The joining surface in the case of a tablet subunit is typically any flat surface on the tablet subunit, but is not limited to such a surface. About ten to twenty seconds are allowed to elapse while some or all of the solvent (alcohol) present in the shellac solution evaporates, leaving a sticky adhesive residue on the tablet subunit. Then the joining surface of the second tablet subunit is brought into contact with the sticky surface of the first subunit. The subunits are held together by light hand pressure for about twenty seconds, and then allowed to dry in the ambient air.
2. An alternate method comprises placing the shellac solution on the joining surface of both subunits at about the same time, manually applying light pressure to both subunits for twenty seconds, and allowing the dosage form to air dry for about one minute to allow evaporation of the solvent.
3. Similar procedures may be following in joining capsules to each other, and utilizing linkers (subunits) to join tablet to tablet, capsule to capsule, and tablet to capsule, creating dosage forms in which the preformed tablets and capsules then function as tablet subunits and capsule subunits.
No limitation is intended in terms of the dilution of the shellac, the choice of specific adhesive, the time or degree of pressure applied to adhesively join the subunits, the time allowed for any solvent to evaporate, the number of subunits joined, etc. The invention expressly contemplates automated or semi-automated methods of adhesively joining the subunits.

1. A solid pharmaceutical dosage form comprising a plurality of adhesively-joined subunits and also comprising one or more of the following:
A. (i) a first inert tablet subunit and (ii) a second active subunit;
B. (i) a first tablet subunit with a pharmacologically inactive layer in which said layer has a mass of at least 20 mg and (ii) a second subunit;
C. a tablet subunit that is provided with a separation mark; or
D. (i) a first tablet subunit and (ii) a second capsule subunit that is adhesively joined to said first tablet subunit.

2. A solid pharmaceutical dosage form as defined in claim 1 which comprises two active subunits.

3. A solid pharmaceutical dosage form as defined in claim 1 in which all subunits contain an active ingredient and a tablet subunit is provided with a separation mark.

4. A solid pharmaceutical dosage form as defined in claim 3 in which said separation mark comprises a score.

5. A solid pharmaceutical dosage form as defined in claim 2 comprising an inert tablet subunit adhesively joined to two active subunits, and said active subunits are not adhesively joined to each other.

6. A solid pharmaceutical dosage form as defined in claim 5 where the inert tablet subunit is provided with a separation mark.

7. A solid pharmaceutical dosage form as defined in claim 7 in which said separation mark comprises a score.

8. A solid pharmaceutical dosage form as defined in claim 1 where an inert tablet subunit is provided with a separation mark.

9. A solid pharmaceutical dosage form as defined in claim 8 where said separation mark comprises a score.

10. A solid pharmaceutical dosage form as defined in claim 1 that comprises an active subunit provided with a separation mark.

11. A solid pharmaceutical dosage form as defined in claim 12 where said separation mark comprises a score.

12. A solid pharmaceutical dosage form as defined in claim 1 wherein the adhesive comprises shellac, a pharmaceutically acceptable acrylic polymer, or hydroxypropyl methylcellulose.

13. A solid pharmaceutical dosage form as defined in claim 1 in which all active subunits contain the same drug or drugs.

14. A solid pharmaceutical dosage form as defined in claim 1 in which two active subunits contain a different drug or drugs.

15. A method of subdividing a solid pharmaceutical dosage form as defined in claim 1 which comprises breaking said dosage form through a tablet subunit while not dissolving, removing, or breaking through said adhesive that joins a plurality of subunits together.

16. A method as defined in claim 15 that involves breaking through a separation mark.

17. A method as defined in claim 16 wherein said separation mark comprises a score.

18. A method of administering a part of an active subunit of a solid pharmaceutical dosage form as defined in claim 1, said method comprising first breaking said pharmaceutical dosage form through an active subunit and then administering said part, said part optionally including one or more additional subunits or parts thereof, to a human patient or other mammal or other animal in need thereof, who then ingests said part of said dosage form enterally.

19. A method of administering an active pharmaceutical ingredient that is present within an active subunit of a solid pharmaceutical dosage form as defined in claim 1 which comprises breaking said dosage form through an inert tablet subunit and then administering a part of said dosage form containing all or part of an active tablet subunit and an inert tablet subunit to a human patient or other mammal or animal in need thereof, who then ingests said part of said dosage form enterally.

20. A method of making a solid pharmaceutical dosage form as defined in claim 1 which comprises:
(a) first, applying adhesive to a first active subunit or inert tablet subunit; and
(b) second, bringing said first active subunit or inert tablet subunit containing adhesive into contact with a second active subunit or inert tablet subunit that optionally is provided with adhesive so that said first subunit and said second subunit adhere to one other, optionally pressing said first and second subunits together.
